(19) United States
(12) Patent Application Publication
(10) Pub. No.: US 2023/0149085 A1
(43) Pub. Date: May 18, 2023

(54) SURGICAL SIMULATION DEVICE

(71) Applicant: VIRTUALISURG, Neuilly-sur-Seine (FR)

(72) Inventors: Nicolas MIGNAN, Neuilly-sur-Seine (FR); David REVERSAT, Neuilly-sur-Seine (FR)

(73) Assignee: VIRTUALISURG, Neuilly-sur-Seine (FR)

(21) Appl. No.: 17/917,374

(22) PCT Filed: Apr. 9, 2021
(86) PCT No.: PCT/FR2021/050633

§ 371 Date: Oct. 6, 2022

(30) Foreign Application Priority Data
Apr. 10, 2020 (FR) FR2003649

(51) Int. Cl. A61B 34/10

(52) U.S. Cl. CPC A61B 34/10; A61B 2034/102; A61B 2090/365

(57) ABSTRACT

A surgical simulation device having a real surgical instrument including a functional element, which can be activated according to at least two distinct operating states, and a computing unit. The device also includes an electronic system connecting the real surgical instrument to the computing unit, and a virtual surgical instrument connected to the computing unit. The virtual surgical instrument has a virtual functional element similar to that of the real surgical instrument. The real surgical instrument provided with the electronic system has substantially the same weight as a corresponding functional surgical instrument, the virtual functional element of the virtual surgical instrument is adapted to be activated according to the same operating states as those of the functional element of the real surgical instrument, and to be aligned, in real time, with the operating state of the real surgical instrument.

### TECHNICAL SCOPE OF THE INVENTION

The present invention relates to the field of medical instruments and equipment. More particularly, the invention relates to a device for surgical simulations.

### TECHNICAL BACKGROUND

It is known in the field of medicine to offer training devices to trainee surgeons. Trainees can of course train on deceased bodies, but these are limited in number. Trainees can also train on living patients under the supervision of a senior surgeon, but this practice poses a risk to the patient. It is therefore essential to provide systems to free surgical learning from the availability of deceased bodies or patients.

Many examples of such systems already exist in the state of the art, as illustrated, for example, in EP 1746558 B1, and WO 2019204615 (A1).

Document EP1746558 B discloses a system for simulating a surgical operation, by a user, on a body, simulated with at least two real instruments. The system comprises a longitudinal track and a plurality of carriages movable along said track. Each carriage has clamping means and means for rotating and longitudinally moving said real instruments. The system also comprises feedback means for receiving and transmitting, to the user's hand, a feedback force from said real instrument with respect to the simulation characteristic, means for recognising a real instrument to be inserted into said clamping means, whereby said real instrument can be fixed within said clamping means to be moved longitudinally and rotated by the user.

Document WO 2019204615 (A1) discloses an apparatus comprising an endoscopy device, and a tracking device adapted to work with a three-dimensional tracking system to track the location and orientation of the endoscopy device in three dimensions in a simulated operating room environment. The apparatus also includes a physical model of a patient's head comprising hard and soft components, and the endoscopy device is configured to be inserted into the physical model to provide haptic feedback of the endoscopic surgery.

Both documents disclose surgical training devices by combining a mechanical system (surgical instruments and/or training consoles) with a sensor system and a display system. The sensors determine the positioning of the instruments used by the operator in relation to the elements of the training console. Data is displayed on a display system to assist the surgical trainee. However, none of these devices allow for real immersion. The conditions of the operating theatre are not reproduced and the trainee cannot experience all the sensations of an operating theatre procedure. The prior art disclosures lack a virtual component to the simulation, in order to significantly approximate the operating conditions in the operating room. The only way to reproduce these conditions in a relevant way is to immerse the operator in a virtual world, while allowing him to manipulate real surgical instruments in order to prepare him as well as possible for the real conditions of the operating room.

Virtual reality is also used to accompany a surgeon during a surgical procedure, as for example illustrated by WO 2017114834 (A1).

Document WO 2017114834 (A1) discloses a control unit provided for a surgical robot system, comprising a robot configured to operate a surgical tool on a patient. The control unit includes a processor configured to transmit live images acquired from the patient to a virtual reality (VR) device for display. The unit processes the input data received from the VR device to determine a target on the patient and determine a path for the surgical tool to reach the target based on the live images and the processed input data; and to transmit control signals to cause the robot to guide the surgical tool to the target via the determined path.

However, when accompanying a surgeon during an operation, it is not a question of recreating the conditions of the operating theatre in order to familiarise a beginner.

The prior art disclosures do not allow the user to manipulate real surgical instruments simultaneously in the physical world and in a virtual world reproducing the operating conditions in the operating room.

It is to these disadvantages that the invention more particularly intends to remedy by proposing a surgical simulation device combining a virtual world with the use of real surgical instruments.

### SUMMARY OF THE INVENTION

This is achieved in accordance with the invention by means of a surgical simulation device, comprising:
a computing unit,
a real surgical instrument, and
a virtual surgical instrument connected to the computing unit,
an electronic system comprising an electronic card and at least one sensor, the electronic system connecting the real surgical instrument to the computing unit, the electronic card and the at least one sensor being integrated into the real surgical instrument by means of at least one specific interface part.

The invention is characterised in that:
the real surgical instrument with the electronics has substantially the same weight as the corresponding functional surgical instrument
the real surgical instrument corresponds to a functional surgical instrument, the functional surgical instrument being intended to be manipulated within the framework of a surgical operation, the functional surgical instrument comprising at least one functional element, the real surgical instrument comprising the same functional element, the at least one functional element being able to be activated according to at least two distinct operating states,
the virtual surgical instrument has the same geometrical characteristics as the real surgical instrument and has a virtual functional element similar to the functional element of the real surgical instrument,
the virtual functional element of the virtual surgical instrument is adapted to be activated in the same operating states as the functional element of the real surgical instrument, and
the operating state of the virtual functional element of the virtual instrument is adapted to be aligned, in real time, with the operating state of the functional element of the real surgical instrument.

Thus, this solution achieves the above-mentioned objective. In particular, providing instruments with at least one sensor and linking each of them to a virtual twin that the operator has in his virtual field of view, significantly increases the realism of the training and almost identically reproduces the operating conditions in an operating theatre. The activation by the operator of the mechanical or electronic functionalities of the real surgical instrument triggers an identical action in the virtual world, i.e. the operating state of the real surgical instrument is instantly reproduced in the virtual world by the virtual surgical instrument. Furthermore, this surgical simulation device allows the connection of a wide variety of surgical instruments (mechanical and/or electronic, small and/or large, rigid and/or flexible).

The surgical simulation device according to the invention may comprise one or more of the following features, taken alone or in combination with each other:
the at least one sensor of the electronic system may constitute a functional element of the real surgical instrument,
the at least one sensor of the electronic system may be for measuring a mechanical capacity of a functional element of the actual surgical instrument,
the at least one sensor of the electronic system may be intended to measure a relative movement of a functional element of the real surgical instrument with respect to an original position,
the real surgical instrument provided with the electronic system may have dimensions, shapes, and a centre of mass substantially identical to those of the functional surgical instrument,
the electronic card, the at least one sensor and the at least one specific interface piece are integrated into the real surgical tool in replacement of at least one electronic component of a set of electronic components of the functional surgical instrument,
the virtual surgical instrument may be adapted to be viewed by the operator on a viewing device connected to the computing unit,
the real surgical instrument may be provided with a haptic device so as to be able to simulate, for the operator, an interaction with a predefined body, of a nature and positioning determined by the computing unit,
a virtual equivalent of the predefined body is adapted to be visualized, by the operator, on the display device,
the real surgical instrument may be provided with a sound feedback system,
the real surgical instrument may be provided with a spatial localization system so that the computing unit can determine, at each instant, the positioning of the real surgical instrument in space with respect to a predefined origin.

### BRIEF DESCRIPTION OF THE FIGURES

Further features and advantages of the invention will become apparent from the following detailed description, for the understanding of which reference is made to the attached drawings in which:

FIG. 1 is a generalized schematic view of the simulation device according to the present invention,

FIG. 2 is a perspective view of a first embodiment of a real surgical instrument according to the invention,

FIG. 3A is a perspective view of a first specific interface part according to the invention,

FIG. 3B is a perspective view of the interface piece of FIG. 3A integrated with a surgical instrument according to the embodiment of FIG. 2,

FIG. 4A is a perspective view of a second specific interface part according to the invention,

FIG. 4B is a perspective view of the interface piece of FIG. 4A integrated with a surgical instrument according to the embodiment of FIG. 2,

FIG. 5 is a perspective view of a virtual surgical instrument according to the invention,

FIG. 6A is a perspective view of a virtual operating theatre at the time of starting an operation,

FIG. 6B is a perspective view of the operating theatre of the previous figure during the operation,

FIG. 7A is an illustration of a virtual screen of the virtual operating theatre of FIGS. 6A and 6B, during operation, more particularly, FIG. 7A is an illustration of a virtual screen in the virtual operating theatre providing access to an interior view of a virtual patient,

FIG. 7B is an illustration of the virtual screen of the previous figure at the end of the operation,

FIG. 8A is a perspective view of a second example of a real surgical instrument according to the invention,

FIG. 8B is a perspective view of the embodiment of the previous figure, in which the specific interface piece is open.

### DETAILED DESCRIPTION OF THE INVENTION

In the present application, the term “to integrate” is used in the dictionary sense of placing something in a set in such a way that it appears to belong to it, that it is in harmony with the other elements of the set. To integrate something into something means to incorporate it, to make it part of a whole.

In the present application, the term “sensor” refers to a device that transforms the state of an observed physical quantity into a usable quantity, such as, for example, an electric voltage, a mercury height, or the deflection of a needle. It is noted that a is at least constituted by a transducer.

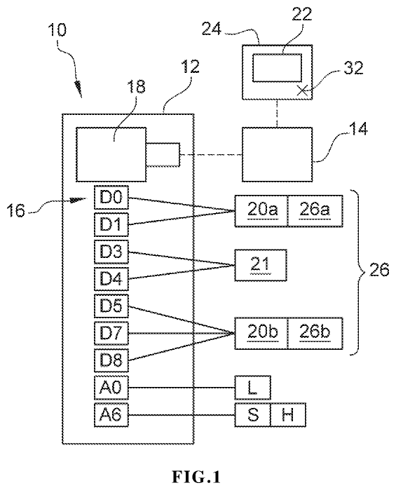

As schematically shown in FIG. 1, a surgical simulation device 10 according to the present invention comprises:
a real surgical instrument (surgical instrument) 12 intended to be manipulated by an operator,
a computing unit 14,
an electronic system 16 comprising an electronic card 18 and at least one sensor 20a, 20b, 21,
a virtual surgical instrument 22 connected to the computing unit 14.

The real surgical instrument 12 is derived from a functional surgical instrument intended to be manipulated in a surgical procedure. Thus, even though the real surgical instrument 12 is not functional in an operating room setting, it reproduces substantially the same physical sensations as a functional instrument when manipulated by an operator in the context of the present invention.

The virtual surgical instrument 22 is made visible to the operator by projection onto a display device 24.

In this case, the operator may be a trainee surgeon.

The display device 24 is, for example, a virtual reality headset. The operator puts on the headset to perform the surgical simulation.

### Real Surgical Instrument

The electronic system 16 connects the surgical instrument 12 to the computing unit 14. The electronic system 16 is integrated with the surgical instrument 12. The electronic system 16 may be integrated into any type of surgical instrument 12, including, for example, foot switches arranged around machines typically present in an operating room (such as an ultrasound scanner, a milling machine or bed), a photopolymerization lamp or a milling speed control unit for example. Furthermore, the electronic system 16 is transparent in size, shape, weight and centre of mass to the operator handling the surgical instrument 12.

The electronic card 18 of the electronic system 16 may for example be a board of the Arduino®, Teensy®, MBed® type. This electronic board 18 can communicate with or without wires (for example according to BLE or WIFI protocols) with the computing unit 14. This computing unit 14 may, for example, be a remote computer or a microcontroller comprising an arithmetic and logic unit and a memory. The electronic card 18 may, for example, be powered by a rechargeable battery (Li—Po, Ni-MH, Li-Ion . . . ), or by a battery. The electronic card 18 also allows direct feedback to the operator on the status of the electronic system 16 of the surgical instrument 12 by means of a multi-coloured LED (for example to indicate that the device 10 is switched on, that the electronic system 16 is well connected to the computing unit 14, that the battery level is low, that the sensors 20a, 20b, 21 are functional, etc.) without having to start a simulation.

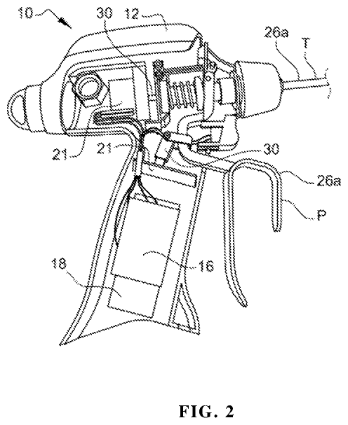

This electronic card 18 has digital and analogue inputs and outputs to retrieve, in real time, information from the sensors 20a, 20b, 21 integrated in the surgical instrument 12. Each sensor 20a, 20b, 21 collects its own functional information. As shown in FIG. 2, all sensors 20a, 20b, 21 are integrated with the surgical instrument 12.

In the case of the present invention, the electronic system 16 comprises three types of sensors 20a, 20b, 21: two types of so-called original sensors (a set of primary original sensors 20a, and a set of secondary original sensors 20b), and one type of so-called additional sensors 21. The primary and secondary original sensors 20a, 20b are elements present on the functional surgical instrument 12 as marketed and used by practitioners in an operating theatre. These original primary and secondary sensors 20a, 20b are disconnected from their basic electronics resulting from their industrial processing and are then integrated into the electronic system 16 of the surgical simulation device 10.

In particular, the original primary and secondary sensors 20a, 20b each constitute a functional element 26 of the surgical instrument 12. A functional element 26 is an element required for the proper operation and/or handling of the surgical instrument 12. Each functional element 26 of the real surgical instrument 12 is identical to the functional element 26 of the corresponding functional surgical instrument. A functional element 26 may be mechanical or electronic. Classically, each functional element 26 may be activated in at least two distinct operating states. This will be explained further below. A functional element 26 may also be primary 26a or secondary 26b. A surgical instrument 12 may thus comprise one or more primary functional elements 26a (electronic or mechanical) and one or more secondary functional elements 26b (electronic or mechanical). A primary functional element 26a may, for example, take the form of an activation handle, button, lever, or touchpad, and it enables the surgical instrument 12 to be operated, activated, and/or controlled, etc. Thus, each primary original sensor 20a forming a primary functional element 26a, allows the computing unit 14 to retrieve an operator action on the surgical instrument 12. The operator performs this action during a surgical simulation for surgical purposes, such as coagulating a vessel, or orienting the effector of the surgical instrument 12. Each secondary original sensor 20b forming a secondary functional element 26b, in turn, provides feedback on the operating status of the surgical instrument 12. A secondary original sensor 20b may, for example, take the form of a buzzer or an LED to, for example, indicate to the operator that a coagulation system is ready or that the surgical instrument 12 is at a certain load level.

Independent of the original sensors 20a, 20b, the additional sensors 21 are added to the functional surgical instrument 12 and are therefore not required for the proper functioning/use of said instrument 12. Each additional sensor 21 is used to measure:
a mechanical capacity of a primary functional element 26a of the actual surgical instrument 12, and/or
a relative movement of a primary functional element 26a of the actual surgical instrument 12 with respect to an original position of said primary functional element 26a,
an orientation of a primary functional element 26a relative to an original position of said primary functional element 26a,
an ambient or internal magnetic field,
an orientation of a primary functional element 26 relative to another primary functional element 26,
a relative position of the surgical instrument 12 in space with respect to a defined reference frame.

An IMU (inertial measurement unit) may, for example, forms an additional sensor 21.

The electronic system 16 can be added to different categories of surgical instruments 12 in a wide range of applications and in all surgical specialties. Classically, two types of functional surgical instruments are considered:
complex surgical instruments,
mechanical surgical instruments.

Complex functional surgical instruments can be electronic and/or mechanical. They may therefore have a wide variety of mechanical and electronic functional elements 26. These mechanical functional elements 26 may take the form of mechanical actuators such as buttons, triggers, activation handles P (see FIG. 2), knobs, dimmers, etc. The mechanical functional elements 26 are primary functional elements 26a. They can be operated by means of a motor or by direct action of the operator. A complex functional surgical instrument also has electronic functional elements 26 such as secondary functional elements 26b such as an LED, for example. Where a complex functional surgical instrument is electronic, it is usually provided with a battery or is connected to an external machine in the operating theatre to enable it to be powered.

The system's electrical power is provided by a 12V/3 A power supply (not shown). The data is transmitted by a wired means of communication (USB 2.0, Ethernet) or by a non-wired means of communication (Wifi, Bluetooth, . . . ).

Specifically, the signal processing performed from each real surgical instrument 12 produces a real-time effect in the virtual reality simulation. Thus, each virtual surgical instrument 22, as a virtual twin, moves and reacts identically to its real model. Each real surgical instrument 12 has a unique identifier which allows the values received to be associated with the corresponding virtual surgical instrument 26, i.e. the correct virtual twin. Each real instrument 12 thus connects to the simulation (TCP, UDP, serial) when it is switched on. Each real instrument 12 then sends its data at a defined frequency to the computing unit 14.

The connection is made between the computing unit 14 and each real surgical instrument 12 via a protocol that can be point-to-point (Unicast) or broadcast (Broadcast or Multicast for example). In all modes, the simulation acts as a data server.

The example in FIG. 2 illustrates the case of a cauteriser.

Mechanical functional surgical instruments do not have electronic functional elements but only mechanical functional elements (primary functional elements 26a). These include surgical retractors, scissors, forceps and needle holders or more complex mechanical systems such as the AMIS® system by Medacta for hip replacement.

As already indicated, each real surgical instrument 12 of the present invention corresponds to a functional instrument and each functional element 26 of the functional surgical instrument corresponds to a functional element 26 of the real surgical instrument 12. Each functional element 26 of the real instrument 12 may be activated, exactly like the corresponding functional element 26 of the functional instrument, in at least two distinct operating states. The sum of the operating states of each of the functional elements 26 of the real surgical instrument 12 provides the operating state of the real surgical instrument 12 itself. For a complex surgical instrument 12, for example, a functional off state and a functional on state can be distinguished. The energised functional state can itself be divided into a resting functional state (the operator does not use the instrument 12) and an activating functional state (the operator activates the instrument 12). Depending on the surgical instruments 12, there may be several functional states of activation, for example if the surgical instrument 12 has a primary functional element 26a that can adopt several speeds, such as the rod T of the surgical instrument 12 of the example shown in FIG. 2. For a mechanical surgical instrument 12, a distinction may, for example, be made between an open functional state and a closed functional state (in the case of forceps, or scissors, for example).

Taking the example shown in FIG. 2, a secondary sensor 21 may for example measure:
the rotation of a shaft T of the actual surgical instrument 12,
a degree of closure of an activation handle P.

Note that the rod T and the activation handle P are each a primary functional element 26a.

The challenge around the sensors is twofold: for the original sensors 20a, 20b the challenge is to disconnect the original electronics to connect it to the electronic system 16 without altering the original functioning of the sensor 20a, 20b, and, for the additional sensors 21, the challenge is to add them without disturbing the functioning of the surgical tool 12.

In addition to complex or mechanical surgical instruments 12, the electronic system 16 may be integrated into a control box present in an operating theatre. This may, for example, be a cold light control box for endoscopic cameras or the control panels of an anaesthesia machine. It is thus possible to recover the actions of a user external to the simulation but present at the operator's side to reproduce his actions in the simulation. For example, in the case of the use of an endoscopic camera in a surgical simulation, it becomes possible to ask an assistant to adjust the intensity of the light of an endoscopic camera while the operator is performing the surgical simulation. To be able to do this, it is necessary to know the degree of light sent by the endoscopic camera and to connect the light block to the simulation. This same type of situation is found in a simulation during which CO₂ is classically injected into the abdominal wall of a patient before the introduction of the tools: indeed, by connecting the CO₂ injector to the electronic system 16, it becomes possible to ensure flow management along the operation and the operator can be accustomed to regularly checking the pressure level, for example.

The notion of a complex surgical instrument 12 covers certain surgical robots such as, for example, a robotic assistance platform handling console which is increasingly used by practitioners.

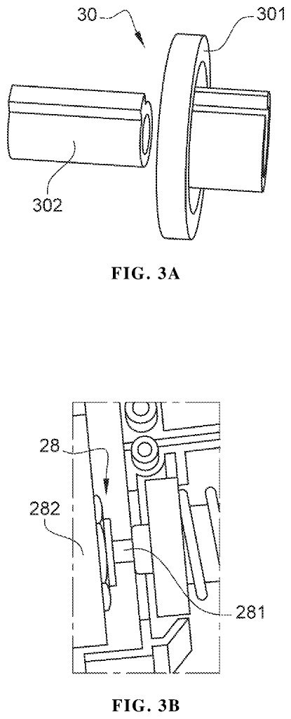

In the example shown in FIGS. 2 and 3B, the rotation of the rod T of the surgical instrument 12 is transmitted via a secondary sensor 21 in the form of an infinitely rotating encoder 28. Generally speaking, an encoder is a hardware or software component that transforms information into a code. A rotational encoder typically comprises a light source, a disc with holes at regular intervals rotating around an axis and an optical sensor. Each time light passes through one of the holes in the disc, an electrical signal is sent. By collecting the signal that passes through each disc, it is possible to know in which direction the axis rotates and by how many degrees. The more holes the disc has, the more precise the angle. In this case, the encoder shaft 281 is coupled to the rod T of the surgical instrument 12. Thus, when the rod T is activated (i.e. rotated), it drives the shaft of the encoder 28. This shaft 281 drives the perforated disc 282 which gives information about the angle of rotation of the rod T.

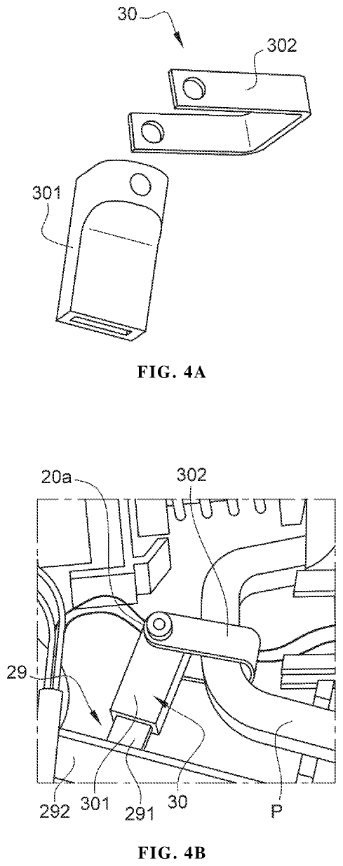

In the example shown in FIGS. 2 and 4B, the degree of closure of the activation handle P is transmitted by a secondary sensor 21 which may, for example, take the form of a rotating or sliding variable resistor (potentiometer) 29 or a force sensor. In general, a type of variable resistor with three terminals, one of which is connected to a slider moving over a block of variable resistor terminated by the other two terminals, is called a potentiometer. This system makes it possible to collect, between the terminal connected to the cursor and one of the other two terminals, a voltage which depends on the position of the cursor and the voltage to which the variable resistance block is subjected, the two terminals corresponding to the maximum and minimum values of the variable resistance block. In the present case, the slider 291 of the linear potentiometer 29 is coupled to the activation handle P. Thus:
when the activation handle P is actuated, the slider 291 of the potentiometer 29 is, along the variable resistance block 292, displaced in an actuation direction and this displacement causes the resistance of the potentiometer 29 to vary in that direction,
when the activation handle P is released, a spring integrated in the actual surgical instrument 12 pushes the activation handle P back to its original state (open) and the slider 291 of the potentiometer is, along the variable resistance block 292, driven in the other direction.

In this way, the minimum and maximum values that can be reached when the activation handle P is opened or closed are known and, by means of a cross product, the percentage of opening or closing of said activation handle P is accessed.

It can be seen from FIGS. 2, 3B and 4B that the electronic card 18 and the sensors 20a, 20b, 21 are integrated into the surgical instrument 12 by means of at least one specific interface part 30. Each specific interface part 30 is obtained by 3D printing.

In the case of the example illustrated in FIGS. 2, 3A and 3B, the connection between the encoder 28 and the rod T of the surgical instrument 12 is enabled by a specific interface piece 30. This specific interface piece 30 is illustrated in FIG. 3A. The specific interface piece 30 of FIG. 3A is in two parts: a first part 301 intended to be glued to the rod T of the surgical instrument 12, and a second part 302 intended to be glued to the shaft of the encoder 28. The shaft of the encoder 28 can be driven by the rod T via a coding system. The specific dimensioning and geometry of the specific interface part 30 linked to the encoder 28 thus makes it possible to ensure that the encoder 28 is driven by the rod T of the surgical instrument 12 without hindering the travel of the rod T during the operation of the surgical instrument 12.

In the case of the example illustrated in FIGS. 2, 4A and 4B, the coupling between the activation handle P of the surgical instrument 12 and the potentiometer 29 is also guaranteed by another specific interface piece 30. As before, this specific interface part 30 comprises two parts: a first part 301 forming a sleeve and intended to be glued around the slider of the potentiometer 29, and a second part 302 forming a hoop and passing around the handle P. The first and second parts 301, 302 of the specific interface part 30 are connected to each other in such a way as to be able to swivel one with respect to the other according to one degree of freedom. The potentiometer 29 is fixedly mounted in the surgical instrument 12. The first part 301 is fixedly mounted on the axis of the potentiometer 29, which itself is slidable relative to the body of the potentiometer. The second part 302 follows the movements of the activation handle P when it is operated by the operator and then transmits these movements to the first part 301 which transmits them to the slider of the potentiometer 29. The information is then sent to the computing unit 14.

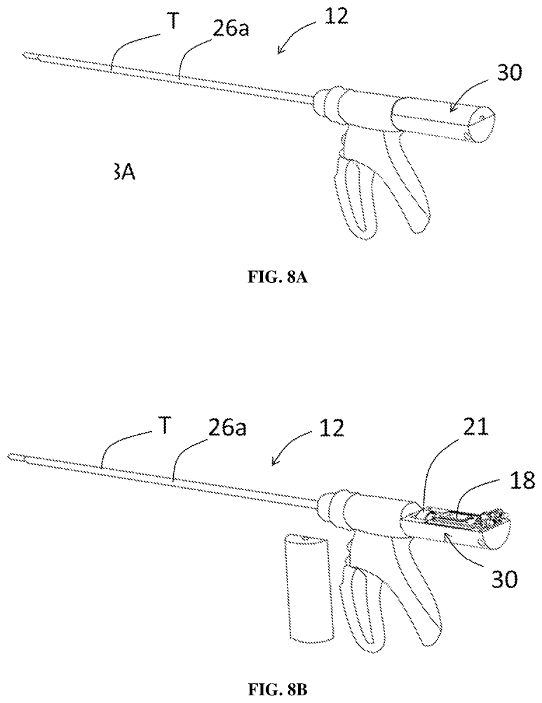

Each additional sensor 21 is added to the surgical instrument 12 in a manner that is transparent to the operator with respect to the functional surgical instrument. In general, the addition of all the sensors 20a, 20b, 21 and the electronic card 18 of the electronic system 16 as well as each of the specific interface parts 30 does not significantly alter the mechanical travel or force required to mechanically actuate each primary and/or secondary functional element(s) 26a and/or 26b of the surgical instrument 12 relative to those of the functional surgical instrument. The physical properties of the surgical instrument 12 (dimensions, shapes, and a centre of mass, etc.) remain, after integration of the electronic system 16, substantially identical to those of the functional surgical instrument obtained from the factory. The challenge, for each surgical instrument 12, is thus to add the measurement system of the electronic system 16 in a substantially transparent manner for the operator so as to preserve all the degrees of freedom of the functional surgical instrument. Indeed, the electronic card 18, each sensor 20a, 20b, 21 and each specific interface part 30 are integrated into the actual surgical tool 12 in replacement of at least one electronic component of a set of electronic components of the functional surgical instrument. In the example illustrated in FIGS. 8A, 8B, the specific interface part 30 is integrated inside the real surgical tool 12 by attaching (docking) it to an end of the surgical instrument 12. This attachment is done in such a way that it does not alter the handling parameters of the surgical tool 12. Thus, in the example shown in FIGS. 8A and 8B, the specific interface piece 30 is attached in continuity with the motor axis of the real surgical tool 12. The final mass of the real surgical tool 12 is maintained substantially the same as that of the functional surgical tool because the electronic card 18, each sensor 20a, 20b, 21 and the specific interface piece 30 are integrated into the real surgical tool 12 in replacement of at least one electronic component of a set of electronic components of the functional surgical instrument, even though they are not integrated at the location where these electronic components were. The specific interface part 30, the electronic card 18, the sensors 20a, 20b, 21 are integrated into the actual surgical tool 12 by attachment (docking) and form a single technical part. Thus, each mass change induced by the addition of a component of the electronic system 16 of the system 10 is compensated by the removal of an electronic component (e.g. a battery) initially present in the functional surgical instrument.

The real surgical instrument 12 may be provided with a sound feedback S. This sound feedback, like what exists in the automotive field to help a user to park, allows to give an indication of the available space around the real surgical instrument 12 or even information on the position of an end of the real surgical instrument 12 in the space and allows to help the operator, at the beginning of learning, to perceive the depth of the working space. This sound feedback S gives the distance between the tip of the instrument and the surgical target. This type of feedback allows additional information to be sent to the user without overloading his visual space so that he can concentrate on his task.

The actual surgical instrument 12 may furthermore be provided with a spatial location system L, so that the computing unit 14 can determine the position of the real surgical instrument 12 in space relative to a predefined origin at any time.

The real surgical instrument 12 may also be provided with a haptic device H so that an interaction with a predefined body can be simulated for the operator. This haptic device H will be described in more detail below. The real surgical instrument 12 may, in addition to the haptic device H, be provided with an overall sensory device, so as to be able to emit, in response to a predefined external signal, a specific sound, light or smell.

All the systems added to the real surgical instrument 12, i.e. the integrated electronic system 16, the haptic system H, the sonar system S and the spatial localization system L, are transparent to the operator: the real surgical instrument 12 does not lose functionality despite the integration of all these systems and the centre of mass of the real surgical instrument 12 is not changed.

### Virtual Surgical Instrument

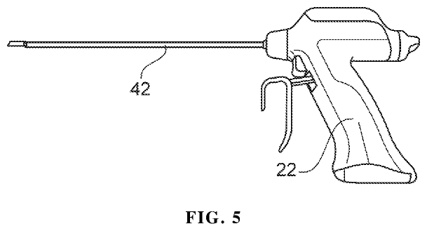

As already mentioned, each real surgical instrument 12 of the present invention is intended to be manipulated by an operator and can be activated in at least two distinct operating states. Furthermore, each real surgical instrument 12 has its own geometrical characteristics. In the surgical simulation device 10 of the present invention, to each real surgical instrument 12, corresponds a virtual surgical instrument 22 (see FIG. 5) having the same geometrical characteristics as those of the corresponding real surgical instrument 12. This is a virtual twin 22 of the real surgical instrument 12. Each virtual surgical instrument 22 can be activated to the same operating states as the corresponding real surgical instrument 12, and the operating state of the virtual instrument 22 aligns, in real time, with the operating state of the corresponding real surgical instrument 12.

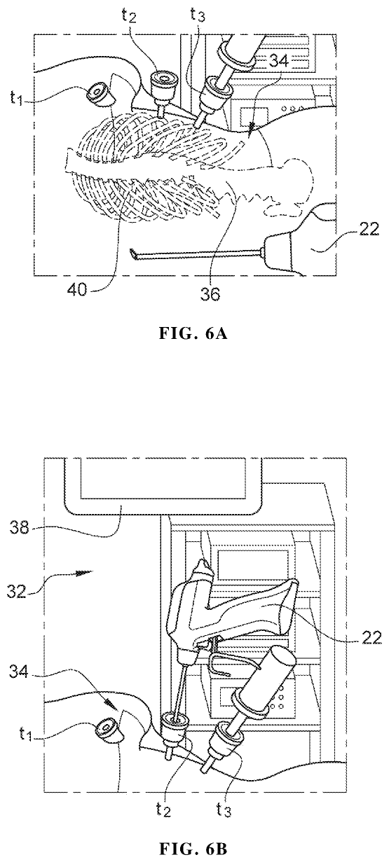

In the example shown in FIG. 5, the virtual surgical instrument 22 is a cauteriser, a twin of the cauteriser shown in FIG. 2. While manipulating the real surgical instrument(s) 12, the operator views each virtual surgical instrument 22 on the viewing device 24 connected to the computing unit 14. In addition to viewing each virtual surgical instrument 22 (in the case of FIG. 6A, a cauteriser and three trocars t1, t2, t3), the operator can view an entire virtual operating room 32 (see FIG. 6B) and even a virtual patient 34 on whom he/she is to perform a surgical simulation. The virtual operating theatre 32 and the virtual patient 34 are stored in the computing unit 14 and made visible to the operator by the latter.

In the example illustrated in FIGS. 6A to 8B, the surgical simulation concerns a thoracic scoliosis correction. In a classical and known way, this surgery is a minimally invasive surgery and the operator is oriented thanks to an image generated by a camera introduced into the patient's body by means of one or more trocars. These trocar(s) are also used, in this case, to guide the real surgical tool 12 towards an image of the organ to be operated on (here, the spine) displayed on a screen. In the case of a surgical simulation, the operator acts, by means of the virtual surgical tool 22 on the virtual organ to be operated 36. In the specific case of the example of minimally invasive surgical simulation illustrated in FIGS. 6A to 8B, the operator sees, on a virtual screen 38, an image 36′ of the virtual organ to be operated 36 (the virtual spine). This virtual screen 38 is part of the virtual operating theatre 32. As seen in FIG. 8A, the operator also sees an image 22′ of the virtual surgical tool 22 on the virtual screen 38. The surgical simulation thus immerses the operator in the real conditions of an operating theatre.

This ‘immersion’ produced by virtual reality combined in real time with real instrumentation from real surgical instruments, accelerates the beneficial effects on the training of the auditory, visual and kinaesthetic memory of the trainee (the user). Through this training, the user will, firstly, actively memorise the gesture and actions to be performed for the simulated procedure. And secondly, passively, the interactions between his different senses will create transferable automatisms in a real context.

Thus, each of the original sensors 20a, 20b or additional sensors 21 added to the functional surgical instrument to measure a degree of rotation, a length of stroke, a percentage of closure, a speed, a rate of battery charge, or a pressure allow these same quantities to be reproduced on the virtual surgical instrument 22. As each sensor is connected to the electronic card 18, which in turn is connected to the computing unit 14, the computing unit 14 can therefore, in real time, reproduce the mechanical operation of each real surgical instrument 12 during the simulation.

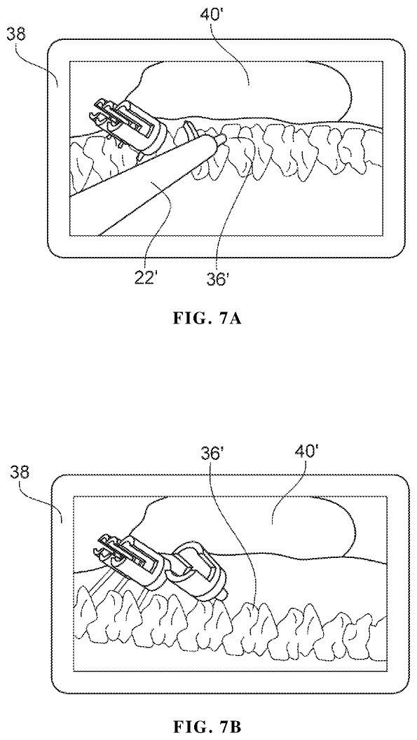

Furthermore, the real surgical instrument 12 may be provided with a haptic device so that an interaction with a predefined body can be simulated for the operator. This predefined body is a virtual body which has a virtual nature and a virtual positioning determined by the computing unit 14. The operator visualises a virtual equivalent of the predefined body as a virtual anatomical object 40 via the display device 24. In this case, since it is a minimally invasive surgery, the operator sees an image 40′ of each anatomical object 40 surrounding the virtual organ to be operated on 36. In FIG. 6A, ribs can be seen, in FIGS. 7A, 7B, an image of a lung can be seen.

Each predefined body therefore simulates a virtual anatomical object 40 in virtual reality. As already mentioned, this virtual anatomical object 40 can be a lung, a liver, a muscle, a bone, etc. The haptic feedback H built into the surgical instrument 12 (complex or mechanical) maximises the realism of the surgical simulation by providing force feedback sensations to the operator. Using a cable system or vibration technology (e.g. an eccentric rotating mass motor (ERM), or a piezoelectric motor, etc.), the palpation or collision of the real surgical instrument 12 (or virtual surgical instrument 22) with a virtual anatomical object 40 in the virtual reality can be felt. The operator may also feel the pulling force of a suture, for example. In the case where the real surgical instrument 12 is provided with an overall sensory device, the sound, light and/or smell emitted in response to the external signal further intensifies the immersive experience.

Thus, the electronic system 16 integrated into the real surgical instrument 12 allows information on the status of the real surgical instrument 12 to be transmitted in real time to its virtual twin 22. Like the real surgical instrument 12, the virtual surgical instrument 22 has at least one virtual functional element 42 (see FIG. 5). This virtual functional element 42 is a twin of the corresponding real functional element 26. Thus, the operating state of the virtual functional element 42 of the virtual instrument 22 is aligned, in real time, with the operating state of each functional element 26 of the real surgical instrument 12. To ensure the performance of the immersive experience, the alignment of the functional state of the corresponding real and virtual surgical instruments 12, 22 (or their corresponding functional elements 26, 42) occurs without any apparent delay to the operator. The integrated electronic system 16 is able to follow the evolution of the functional states of the real surgical instrument 12 according to the full functionality of the latter, respecting the ergonomics and geometrical characteristics of the latter, and to be sufficiently miniaturised so as not to add to the weight of the real surgical instrument 12 and so as not to impede the operator during the surgical simulation.

It is noted that the surgical simulation device 10 according to the present invention allows an operator to manipulate simultaneously in the physical world and in the virtual world real surgical instruments 12. Thus, each real surgical instrument 12 used in the operative steps of a surgical procedure is connected in real time to a virtual reality comprising a virtual surgical instrument 22 corresponding to each real surgical instrument 12.

The technology developed by the present invention thus provides a perfect match between the virtual world and the real world, without which the skills acquired in simulation will be insufficient and approximate.

1-11. (canceled)

12. A surgical simulation device, comprising:
a computing unit,
a real surgical instrument, and
a virtual surgical instrument connected to the computing unit, and
an electronic system comprising an electronic card and at least one sensor, the electronic system connecting the real surgical instrument to the computing unit, the electronic card and the at least one sensor being integrated into the real surgical instrument by means of at least one specific interface part,
wherein:
the real surgical instrument with the electronics has substantially the same weight as the corresponding functional surgical instrument,
the real surgical instrument corresponds to a functional surgical instrument, the functional surgical instrument being intended to be manipulated within the framework of a surgical operation, the functional surgical instrument comprising at least one functional element, the real surgical instrument comprising the same functional element, the at least one functional element being able to be activated according to at least two distinct operating states,
the virtual surgical instrument has the same geometrical characteristics as the real surgical instrument and has a virtual functional element similar to the functional element of the real surgical instrument,
the virtual functional element of the virtual surgical instrument is adapted to be activated in the same operating states as the functional element of the real surgical instrument, and
the operating state of the virtual functional element of the virtual instrument is adapted to be aligned, in real time, with the operating state of the functional element of the real surgical instrument.

13. The surgical simulation device according to claim 12, wherein the at least one sensor of the electronic system constitutes a functional element of the real surgical instrument.

14. The surgical simulation device according to claim 12, wherein the at least one sensor of the electronic system is for measuring a mechanical capacity of a functional element of the actual surgical instrument.

15. The surgical simulation device according to claim 12, wherein the at least one sensor of the electronic system is intended to measure a relative movement of a functional element of the real surgical instrument with respect to an original position.

16. The surgical simulation device according to claim 12, wherein the real surgical instrument provided with the electronic system has dimensions, shapes, and a centre of mass substantially identical to those of the functional surgical instrument.

17. The surgical simulation device according to claim 12, wherein the electronic card, the at least one sensor and the at least one specific interface piece are integrated into the real surgical tool in replacement of at least one electronic component of a set of electronic components of the functional surgical instrument.

18. The surgical simulation device according to claim 12, wherein the virtual surgical instrument is adapted to be viewed by the operator on a viewing device connected to the computing unit.

19. The surgical simulation device according to claim 12, wherein the real surgical instrument is provided with a haptic device so as to be able to simulate, for the operator, an interaction with a predefined body, of a nature and positioning determined by the computing unit.

20. The surgical simulation device according to claim 18, wherein a virtual equivalent of the predefined body is adapted to be visualized, by the operator, on the display device.

21. The surgical simulation device according to claim 12, wherein the real surgical instrument is provided with a sound feedback system.

22. The surgical simulation device according to claim 12, wherein the real surgical instrument is provided with a spatial localization system so that the computing unit can determine, at each instant, the positioning of the real surgical instrument in space with respect to a predefined origin.
